(19) United States
(12) Patent Application Publication
(10) Pub. No.: US 2026/0083183 A1
(43) Pub. Date: Mar. 26, 2026

(54) Breast fixing brassiere for radiation therapy having a pair of breast covers with incision element

(71) Applicant: UIF (University Industry Foundation), Yonsei University, Seoul (KR)

(72) Inventor: Seungkwon Ahn, Seoul (KR)

(21) Appl. No.: 19/036,299

(22) Filed: Jan. 24, 2025

(30) Foreign Application Priority Data
Sep. 26, 2024 (KR) 10-2024-0130458

(51) Int. Cl. A41C 3/00; A61N 5/10

(52) U.S. Cl. CPC A41C 3/0064; A61N 5/10; A61N 2005/1097

(57) ABSTRACT

Disclosed herein is a breast fixation bra for radiation treatment having at least one cutout portion, the breast fixation bra including: a body part provided to cover the breasts in the lateral direction; a pair of breast cover parts provided to protrude on both sides of the central portion of the body part in order to correspond to the shapes of the breasts; at least one cutout portion provided in a shape in which at least a portion is cut out from a corresponding one of the breast cover parts; and at least one lateral support member provided across at least one of the breast cover parts with one end thereof coupled to the central portion of the body part and the other end coupled to a side portion or the rear support member of the body part, and configured to support a breast accommodated in the cutout portion.

### CROSS-REFERENCE TO RELATED APPLICATION

This application claims the benefit of Korean Patent Application No. 10-2024-0130458 filed on Sep. 26, 2024, which is hereby incorporated by reference herein in its entirety.

### BACKGROUND

### 1. Technical Field

The present invention relates to a breast fixation bra for radiation treatment. More particularly, the present invention relates to a breast fixation bra for radiation treatment provided with a pair of breast cover parts having at least one cutout portion.

### 2. Description of the Related Art

Treatment methods for breast cancer include surgical resection, radiation treatment, chemotherapy, and hormone therapy, and the treatment method varies depending on the stage of breast cancer. In the early stage of breast cancer, radiation treatment is generally performed along with lumpectomy.

When radiation treatment for breast cancer is planned, the radiation doses radiated onto the contralateral breast and the lungs and heart located inside the chest wall, the reproducibility of a patient posture, and a patient fixation effect need to be taken into consideration as important factors.

In order to radiate appropriate doses of radiation onto the breast, the chest wall, and the loco-regional lymphatics during radiation treatment, the use of a patient fixation device is required to ensure the reproducibility of an exact patient posture during treatment.

There are variables when the three- or four-dimensional radiation treatment that is generally used is planned. The unclearness of the boundary between a tumor and sensitive tissue (organ delineation), the shape and boundary of a beam, biological considerations, the heterogeneity of the tumor dose, and the movement of a patient posture need to be taken into consideration.

Accordingly, the present applicant proposed a “Breast Fixation Bra for Radiation Treatment” under Korean Patent No. 10-2173342. However, the invention proposed in Korean Patent No. 10-2173342 is different from the present invention in that each breast cover part is not provided with a cutout portion and a vertical support member for the adjustment of a breast position.

Radiation treatment for the breasts is usually performed one breast at a time, even when both breasts require radiation treatment.

It is important that the breast subjected to radiation treatment is continuously maintained in a position and shape appropriate for radiation treatment.

In addition, when radiation treatment is performed on one breast, it is important to minimize the effect of radiation on the other breast not subjected to radiation treatment by separating the other breast as much as possible.

Therefore, the present invention provides a configuration in which a cutout portion is formed in each of a pair of breast cover parts, so that the breast subjected to radiation treatment maintains a position and shape appropriate for radiation treatment through protrusion through the cutout portion and the other breast protruding through the cutout portion is separated as much as possible.

### SUMMARY

A breast fixation bra for radiation treatment provided with a pair of breast cover parts having at least one cutout according to the present invention has the following objects:
First, the breast fixation bra is intended to have at least one cutout in a breast cover part.
Second, the breast fixation bra is intended to maintain the position and shape of the breast that is subjected to radiation treatment.
Third, the breast fixation bra is intended to separate the breast not subjected to radiation treatment as much as possible.
Fourth, the breast fixation bra is intended to more tightly fix and support the position of the breast accommodated in a cutout in the lateral direction.
Fifth, the breast fixation bra is intended to provide a bra made of a material that does not affect radiation treatment.

The objects of the present invention are not limited to those mentioned above, and other objects not mentioned will be clearly understood by those skilled in the art from the following description.

According to an aspect of the present invention, there is provided a breast fixation bra for radiation treatment provided with a pair of breast cover parts having at least one cutout portion, the breast fixation bra including: a body part provided to cover the breasts in the lateral direction; a pair of breast cover parts provided to protrude on both sides of the central portion of the body part in order to correspond to the shapes of the breasts; at least one cutout portion provided in a shape in which at least a portion is cut out from a corresponding one of the breast cover parts; and at least one lateral support member provided across at least one of the breast cover parts with one end thereof coupled to the central portion of the body part and the other end thereof coupled to a side portion or the rear support member of the body part, and configured to support a breast accommodated in the cutout portion.

The present invention may further include at least one vertical support member provided on at least one of the pair of breast cover parts with one end thereof coupled to one side of the breast cover part and the other end thereof coupled to any one of the rear support member of the body part and a rear connection member protruding upward from the rear support member via the shoulder.

The present invention may further include at least one diagonal support member provided across at least one of the pair of breast cover parts with one end thereof coupled to a side portion of the breast cover part and the other end thereof coupled to any one of the vertical support member coupled to the other breast cover part, the rear support member, and a rear connection member protruding upward from the rear support member.

In the present invention, the lateral support member may include lateral support members that are symmetrically arranged across the breast cover parts, respectively.

In the present invention, the lateral support member may include a plurality of lateral support members that are provided across a preset one of the breast cover parts in an overlapping manner.

In the present invention, when two lateral support members are provided across one of the breast cover parts in an overlapping manner, one end of the lateral support member provided on a lower side may be coupled to the side portion or rear support member of the body part, and one end of the lateral support member provided on an upper side may be coupled to the rear support member of the body part.

In the present invention, the cutout portion may be cut out along an overall breast parenchyma line.

In the present invention, the cutout portion may be cut out along an overall breast parenchyma line, but at least a portion thereof may be cut out through a portion outside the breast parenchyma line.

In the present invention, the cutout portion may have an area outside the center line of each of the breast cover parts that is larger than an area inside the center line.

In the present invention, the cutout portion may not include a portion corresponding to the nipple.

In the present invention, the vertical support member may be provided in a structure in which the binding level thereof can be adjusted, and, when the breast accommodated in the breast cover part is moved, the vertical support member may be adjusted to a loose binding level so that the accommodated breast can smoothly move down outward into the cutout portion through free fall under gravity.

In the present invention, the structure in which the binding level can be adjusted may be any one of a Velcro binding structure and a hook binding structure.

In the present invention, a through hole may be provided in the central portion of the body part, and may allow both the breasts accommodated in the bra to be separated, and moved and then fixed.

In the present invention, a part or all of the one end of the lateral support member may be coupled to the edge of the through hole.

In the present invention, at least one of the body part, the pair of breast cover parts, and the lateral support member may be made of an air-equivalent material woven in a mesh shape.

In the present invention, the vertical support member may be made of an air-equivalent material woven in a mesh shape.

In the present invention, the diagonal support member may be made of an air-equivalent material woven in a mesh shape.

According to another aspect of the present invention, there is provided a breast fixation bra for radiation treatment provided with a pair of breast cover parts having at least one cutout portion, the breast fixation bra including: a body part provided to cover the breasts in the lateral direction; a pair of breast cover parts provided to protrude on both sides of the central portion of the body part in order to correspond to the shapes of the breasts; at least one cutout portion provided in a shape in which at least a portion is cut out from a corresponding one of the breast cover parts; at least one vertical support member provided on at least one of the pair of breast cover parts with one end thereof coupled to one side of the breast cover part and the other end thereof coupled to any one of the rear support member of the body part and a rear connection member protruding upward from the rear support member via the shoulder; at least one lateral support member provided across at least one of the breast cover parts with one end thereof coupled to the central portion of the body part and the other end thereof coupled to a side portion or the rear support member of the body part, and configured to support a breast accommodated in the cutout portion; and at least one diagonal support member provided across at least one of the pair of breast cover parts with one end thereof coupled to a side portion of the breast cover part and the other end thereof coupled to any one of the vertical support member coupled to the other breast cover part, the rear support member, and the rear connection member protruding upward from the rear support member.

The breast fixation bra for radiation treatment provided with a pair of breast cover parts having at least one cutout portion according to the present invention has the following advantages:
First, at least one cutout portion is provided in the breast cover part, so that an advantage is achieved in that the position and posture of the breast protruding through the cutout portion can be more easily controlled.
Second, the cutout portion, the lateral support member, the vertical support member, and the diagonal support member are utilized, so that an advantage is achieved in that the position and shape of the breast that is subjected to radiation treatment can be appropriately maintained.
Third, the cutout portion, the lateral support member, the vertical support member, and the diagonal support member are utilized, so that an advantage is achieved in that the breast that is not subjected to radiation treatment is separated as much as possible.
Fourth, the plurality of lateral support members are provided in an overlapping manner, so that an advantage is achieved in that the breast moved into the cutout portion is further separated from the breast subjected to radiation treatment and the position of the breast is more tightly fixed and supported.
Fifth, when the cutout portion is cut out, the cutout portion is cut out up to the breast parenchyma line or at least partially outside the breast parenchyma line by taking into consideration the breast parenchyma, so that an advantage is achieved in that the breast that is not subjected to radiation treatment can be easily moved into the cutout portion.
Sixth, at least one main component of the bra is made of an air-equivalent material woven in a mesh shape, so that an advantage is achieved in that wearing the bra does not affect radiation treatment.

The advantages of the present invention are not limited to those mentioned above, and other advantages not mentioned above may be clearly understood by those skilled in the art from the following description.

### BRIEF DESCRIPTION OF THE DRAWINGS

The above and other objects, features, and advantages of the present invention will be more clearly understood from the following detailed description taken in conjunction with the accompanying drawings, in which:

FIG. 1 shows an embodiment of a general radiation treatment device, in which the radiation generated from the radiation treatment device is radiated onto a lesion site of the breast of a patient (not shown) who is lying on a couch;

FIG. 2(a) is a schematic diagram of a conventional breast fixation bra, which shows a bra without a cutout portion in a breast cover part, and FIG. 2(b) is a rear view of a patient who wears the conventional breast fixation bra;

FIG. 3 shows the basic structure of a bra before a pair of breast cover parts are provided with at least one cutout portion;

FIG. 4 shows a first cutout embodiment of a cutout portion according to the present invention, in which approximately the overall portion of each of breast cover parts is cut out;

FIG. 5 shows a second cutout embodiment of the cutout portion according to the present invention, in which at least a portion of the cutout portion is cut out up to a portion outside a breast parenchyma line;

FIG. 6 shows a third cutout embodiment of the cutout portion according to the present invention, in which the cutout area of the cutout portion has an area outside the center line of each of breast cover parts that is larger than an area inside the center line;

FIG. 7 shows a fourth cutout embodiment of the cutout portion according to the present invention, in which the cutout area of the cutout portion extends outside a breast parenchyma line and is cut out up to a body part;

FIG. 8 shows an embodiment in which one breast cover part is provided with one lateral support member;

FIG. 9 shows an embodiment in which one lateral support member is provided across each of breast cover parts;

FIG. 10 shows an embodiment in which two lateral support members are provided across one breast cover part in an overlapping manner;

FIG. 11 shows an embodiment in which a diagonal support member is provided across one breast cover part;

FIG. 12 shows an embodiment in which a diagonal support member is provided across each of both breast cover parts;

FIG. 13 shows an embodiment of a breast parenchyma line;

FIG. 14 shows an embodiment in which a cutout portion is cut out up to a body part, which shows a state before one lateral support member covers a cutout portion;

FIG. 15 shows a state after the lateral support member covers and is coupled to the cutout portion in FIG. 14;

FIG. 16 is a front view of FIG. 15, which shows an embodiment in which lateral support members are provided across both breast cover parts, respectively;

FIGS. 17(a) and 17(b) are views showing an embodiment in which an end of a vertical support member according to the present invention is bound to a Velcro fastening portion that is provided on a rear connection member protruding from a rear support member, wherein FIG. 17(b) is an enlarged view of the dotted circular portion of FIG. 17(a);

FIG. 18 shows a state in which one breast cover part becomes loose as it changes from state (X) of FIG. 17 to state (Y) thereof; and

FIGS. 19 and 20 show an embodiment in which a lateral support member and diagonal support member Velcro processed at their ends are bound to a fastening connection member in a Velcro fastening manner, so that tight and loose binding is selectively implemented.

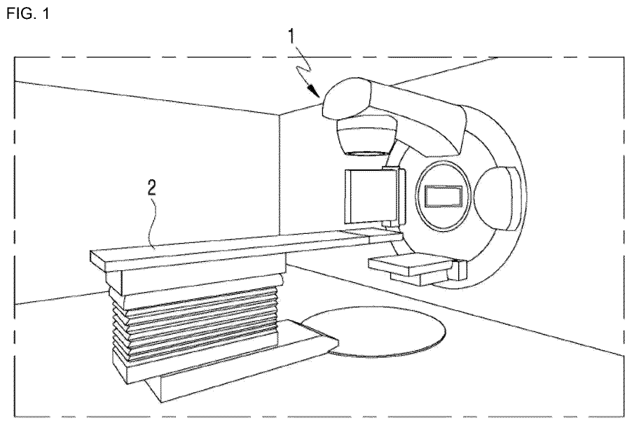

### DETAILED DESCRIPTION

Embodiments of the present invention will be described with reference to the accompanying drawings below so that a person skilled in the art to which the present invention pertains can easily practice the present invention. As can be easily understood by a person skilled in the art, the embodiments to be described below may be modified in various forms without departing from the concept and scope of the present invention. Wherever possible, identical or similar parts are denoted using the same reference numerals throughout the drawings.

The technical terms used herein are used merely to illustrate specific embodiments and are not intended to limit the invention. The singular forms used herein include plural forms unless the phrases clearly indicate the opposite.

The term “include” or its derivative used herein is intended to specify specific features, regions, integers, steps, operations, elements and/or components, but is not intended to exclude the presence or addition of one or more other features, regions, integers, steps, operations, elements, components and/or groups thereof.

All the terms including the technical terms and the scientific terms used herein have the same meanings as generally understood by those having ordinary skill in the art to which the present invention pertains. The terms defined in dictionaries are additionally interpreted as having meanings consistent with the related art literature and the presently disclosed content, and are not interpreted as having ideal or excessively formal meanings unless defined.

The terms regarding directions used herein, such as forward/backward/left/right, up/down, and vertical/horizontal, may be interpreted based on the directions illustrated in the drawings.

More specifically, in the present specification, as for directional expressions, based on the state in which a patient stands upright, the vertical direction is defined as the longitudinal direction of the patient, the lateral direction is defined as the left-right direction of the breast, and the diagonal direction is defined as the direction from the left breast to the right shoulder.

Meanwhile, the breast fixation bra for radiation treatment according to the present invention is used in a situation where a patient is lying on a couch (a bed), especially in a situation where the breast that is not subjected to radiation treatment protrudes through a cutout portion and moves down outward under gravity.

Accordingly, the definitions of directions are made based on the state in which a patient stands upright, and the descriptions of functions are given based on the state in which a patient is lying down and subjected to radiation treatment.

In the present specification, the expressions “pair of breast covering parts” and “each of breast cover parts” are used. It is noted that both expressions are equivalent to each other and are used separately to facilitate descriptions of the invention.

The present invention will be described with reference to the drawings below. For reference, the drawings may be partially exaggerated to describe the features of the present invention. In this case, it is preferable to perform interpretation in light of the overall purport of the present specification.

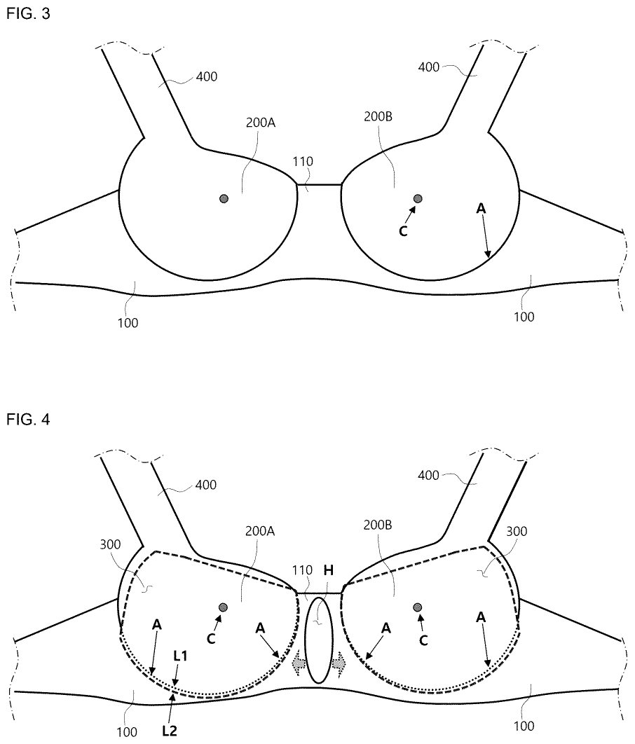

FIG. 3 shows the basic structure of a bra before a pair of breast cover parts are provided with at least one cutout portion. The bra according to the present invention may be implemented in various main embodiments depending on the technical configuration added to the bra of FIG. 3.

A first main embodiment has at least one cutout portion and at least one lateral support member as its essential components, and is an embodiment in which a body part 100, a pair of breast cover parts 200A and 200B, at least one cutout portion 300, and at least one lateral support member 500 are provided. This is an embodiment that embraces bras without shoulder straps (vertical support members).

The breast fixation bra for radiation treatment provided with a pair of breast cover parts having at least one cutout portion according to the present invention includes: the body part 100 provided to cover the breasts in the lateral direction; the pair of breast cover parts 200A and 200B provided to protrude on both sides of the central portion 110 of the body part 100 in order to correspond to the shapes of the breasts; the cutout portion 300 provided in a shape in which at least a portion is cut out from a corresponding one of the breast cover parts 200A and 200B; and the lateral support member 500 provided across at least one of the breast cover parts 200A and 200B with one end thereof coupled to the central portion 110 of the body part 100 and the other end thereof coupled to a side portion 120 or the rear support member 130 of the body part 100, and configured to support the breast accommodated in the cutout portion 300.

A second embodiment has at least one additional vertical support member as its main components, and is an embodiment in which a body part 100, a pair of breast cover parts 200A and 200B, at least one cutout portion 300, at least one vertical support member 400, and at least one lateral support member 500 are provided (see FIGS. 8 to 10).

In the present invention, there may be further provided the vertical support member 400 provided on at least one of the pair of breast cover parts 200A and 200B with one end thereof coupled to one side of the breast cover part 200A or 200B and the other end thereof coupled to the rear support member 130 of the body part 100 via the shoulder.

A third embodiment has at least one additional diagonal support member as its main component, and is an embodiment in which at least one diagonal support member 600 is provided in each of the first and second embodiments (see FIGS. 11 and 12).

In the present invention, there may be further provided the at least one diagonal support member 600 provided across at least one of the pair of breast cover parts 200A and 200B with one end thereof coupled to a side portion 120 of the breast cover part 200A or 200B and the other end thereof coupled to any one of the vertical support member 400 coupled to the other breast cover part 200A or 200B, a rear support member 130, and a rear connection member 131 protruding upward from the rear support member 130.

The cutout portion 300 according to the present invention will be described in more detail below.

The cutout portion 300 according to the present invention may be provided in a shape in which at least a portion is cut out from a corresponding one of the breast covers 200A and 200B.

In the case of the breast that is subjected to radiation treatment, the conventional breast cover part formed in the shape of a cover sometimes obstructs the maintenance of the position and shape of the breast appropriate for radiation treatment. Accordingly, the present invention may perform the function of protruding the breast from the breast cover part 200 by forming the cutout portion 300 and then maintaining the position and shape of the breast appropriate for treatment by utilizing the lateral support member 500. In connection with the maintenance of the position and shape of the breast that is subjected to radiation treatment, the diagonal support member 600 may generally perform a main function, and the lateral support member 500 may also perform an auxiliary function when necessary.

Meanwhile, while a patient is lying on a couch (a bed) for radiation treatment, the patient's normal breast or the breast not subjected to radiation treatment may move down outward from the chest under gravity. The conventional bra has a structure that prevents a patient's normal breast or the breast not subjected to radiation treatment from moving down outward under gravity due to the structure of the breast cover.

Accordingly, even in the case of the other breast that is not subjected to radiation treatment, the present invention may perform the function of protruding the breast from the breast cover part 200 by forming the cutout portion 300 and further separating the freely fallen breast and then maintaining the separated state by utilizing the lateral support member 500.

The cutout portion 300 according to the present invention may be utilized depending on whether treatment is performed on each breast, so that the function of separating the breast subjected to radiation treatment and the breast not subjected to radiation treatment as much as possible can be performed.

The bra according to the present invention may be implemented in various embodiments depending on the structure of the cutout portion 300 provided in the bra of FIG. 3.

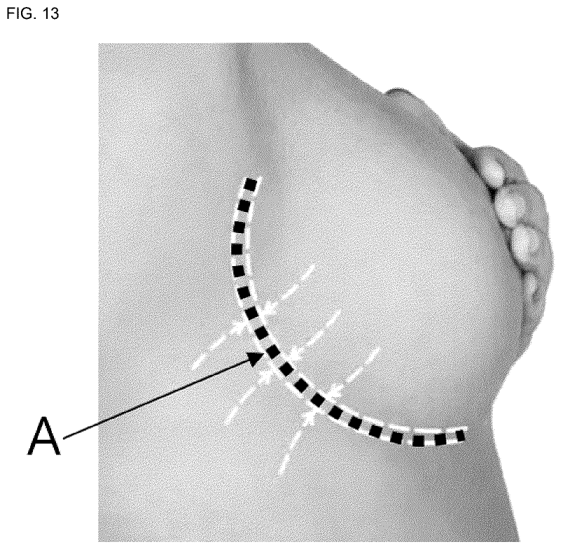

In a first cutout embodiment, a cutout portion 300 according to the present embodiment may be cut out along an overall breast parenchyma line A. FIG. 4 shows a first cutout embodiment of the cutout portion 300 according to the present invention, in which approximately the overall portion of each of breast cover parts 200A and 200B is cut out. As shown in FIG. 4, the black dotted line L1 corresponds to the breast parenchyma line A, and the area of the blue dotted line L2 indicates the area of the cutout portion 300 that is cut out along the breast parenchyma line A. FIG. 13 shows one embodiment of the breast parenchyma line A.

In a second cutout embodiment, a cutout portion 300 according to the present embodiment may be cut out along an overall breast parenchyma line A, but at least a portion thereof may be cut out through a portion outside the breast parenchyma line A.

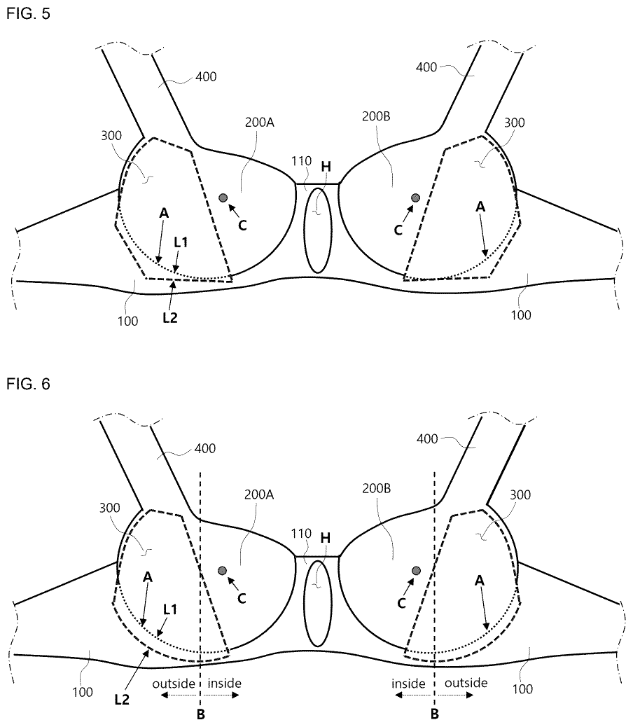

FIG. 5 shows the second cutout embodiment of the cutout portion 300 according to the present invention. As for the blue dotted line L2 of the cutout portion 300, at least a portion thereof is cut out up to a portion outside the breast parenchyma line A.

As shown in FIG. 5, it can be seen that a portion of the blue dotted line L2 coincides with the black breast parenchyma line A, but another portion of the blue dotted line L2 is cut outside the breast parenchyma line A of the black dotted line L1.

Only when the breast parenchyma line A is cut may the breast parenchyma smoothly move down outward under gravity. Furthermore, when a cut is made outside the breast parenchyma line A, the breast parenchyma may move down outward more smoothly.

In a third cutout embodiment, the cutout portion 300 according to the present invention may have an area cut out outside the center line B of the breast cover part that is larger than an area cut out inside the center line B.

FIG. 6 shows the third cutout embodiment of the cutout portion 300 according to the present invention, in which the cutout area of the cutout portion 300 has an area outside the center line B of each of breast cover parts 200A and 200B that is larger than an area inside the center line B.

As shown in FIG. 6, a cutout area L2 is provided inside the center line B, and a larger cutout area L2 is provided outside the center line B. The reason for this is that, especially for the breast that is not subjected to radiation treatment, the cutout area outside the center line B contributes more to the movement of the breast than the cutout area inside the center line B.

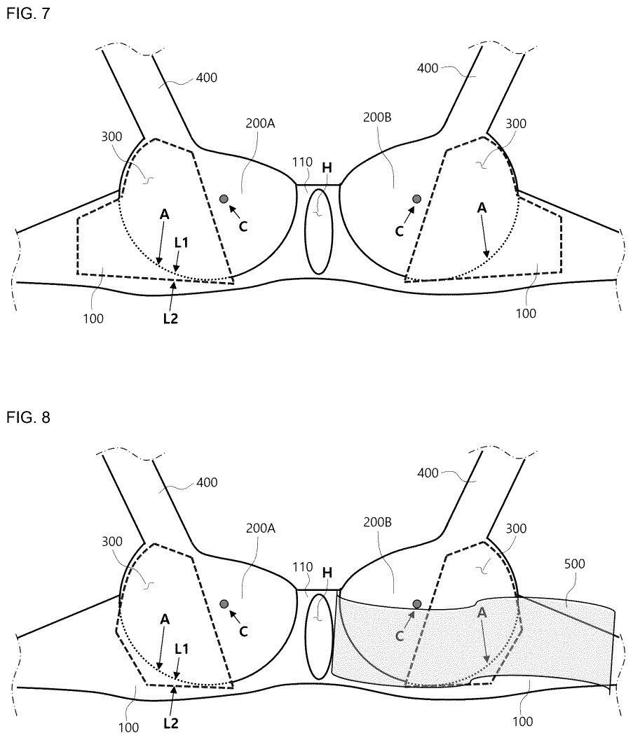

In a fourth cutout embodiment, the cutout portion 300 according to the present invention may be cut out up to a side portion 120 of a body part 100. FIG. 7 shows the fourth cutout embodiment of the cutout portion 300 according to the present invention, in which the cutout area of the blue dotted line L2 of a cutout portion 300 extends outside a breast parenchyma line A and is cut out up to the body part 100.

Meanwhile, respecting the privacy of a female patient is also a significantly important medical courtesy. Accordingly, it is preferable that the cutout portion 300 according to the present invention does not include a portion corresponding to the nipple C. As for the embodiments of FIGS. 5 to 7, it can be seen that the nipple C does not belong to the cutout area of the cutout portion 300 but is covered with each of breast cover parts 200A and 200B.

The lateral support member 500 according to the present invention will be described below.

The lateral support member 500 according to the present invention may be provided across at least one of breast cover parts 200A and 200B with one end thereof coupled to the central portion 110 of the body part 100 and the other end thereof coupled to a side portion 120 or the rear support member 130 of the body part 100, and may be configured to support the breast accommodated in the cutout portion 300.

The lateral support member 500 according to the present invention may be implemented in various embodiments depending on the arrangement position thereof and whether it has an overlap.

A lateral support member 500 according to a first lateral embodiment corresponds to an embodiment in which the lateral support member 500 is provided only across one breast cover part, as shown in FIG. 8.

FIG. 8 shows an embodiment in which one breast cover part 200B is provided with one lateral support member 500. When a separate breast support device is used for the breast that is subjected to radiation treatment, the lateral support member 500 may be provided for a breast cover part that is not subjected to radiation treatment.

Lateral support members 500 according to the second lateral embodiment may symmetrically be arranged across the breast cover parts 200, respectively.

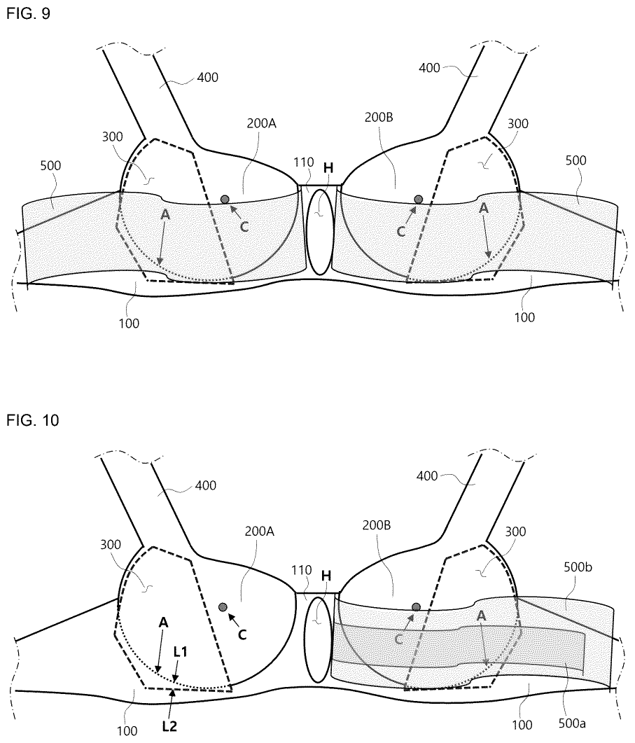

FIG. 9 shows an embodiment in which one lateral support member 500 is provided across each of breast cover parts 200A and 200B. In the case of the breast that is subjected to radiation treatment, the lateral support member may be used for the function of maintaining the position and shape of the breast appropriate for radiation treatment. In the case of the breast that is not subjected to radiation treatment, the lateral support member may be used for the function of moving the breast outward as much as possible and then maintaining the state.

A lateral support member 500 according to a third lateral embodiment corresponds to an embodiment in which a plurality of lateral support members are provided across a preset breast cover part in an overlapping manner. When two lateral support members 500 are provided across one breast cover part in an overlapping manner, one end of the lateral support member 500 provided on the lower side may be coupled to a side portion 120 or the rear support member 130 of the body part 100, and one end of the lateral support member 500 provided on the upper side may be coupled to the rear support member 130 of the body part 100.

This may be applied to each of the first and second lateral embodiments.

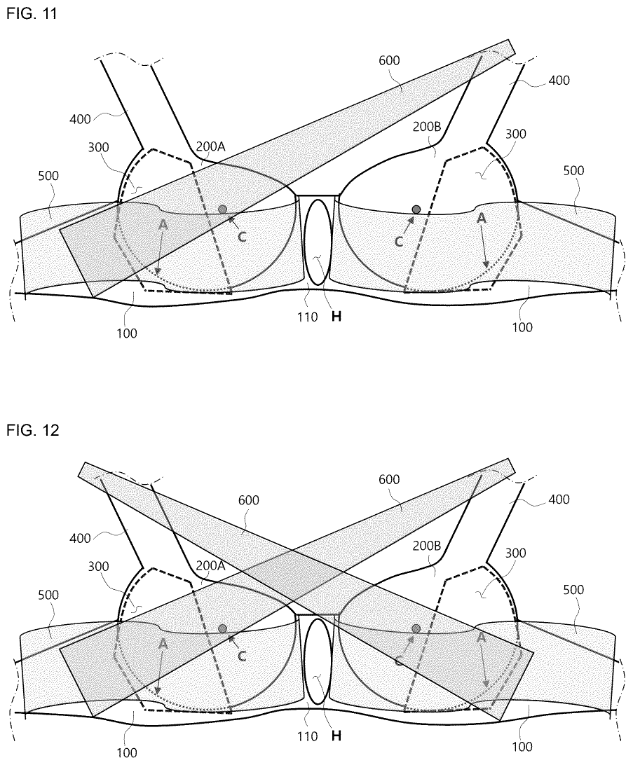

FIG. 10 shows an embodiment in which two lateral support members 500a and 500b are provided across one breast cover part 200B in an overlapping manner. For example, the breast protruding through the cutout part 300 primarily moves down outward in a free-falling manner. Then, the breast is secondarily moved further outward using the lateral support member 500a provided on the lower side and the lateral support member 500a is coupled to the side portion 120 of the body part 100. Furthermore, the breast may be tertiarily moved further outward using the lateral support member 500b provided on the upper side and the lateral support member 500b is coupled to the other side portion 120 or the rear support member 130 of the body part 100.

The vertical support member 400 according to the present invention will be described below.

The vertical support member 400 according to the present invention may be provided on at least one of a pair of breast cover parts 200A and 200B with one end thereof coupled to one side of the breast cover part 200A or 200B and the other end thereof coupled to any one of the rear support member 130 of the body part 100 and a rear connection member 131 protruding upward from the rear support member 130 via the shoulder.

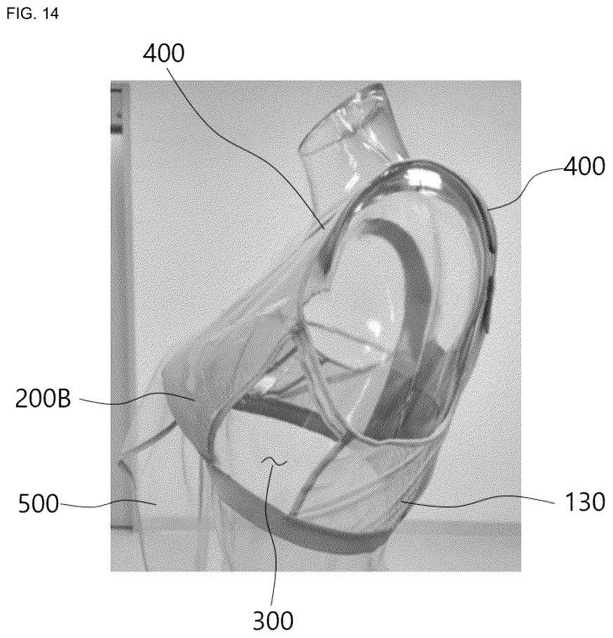

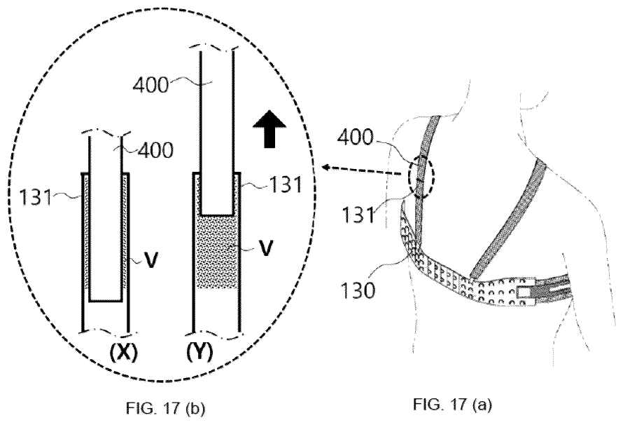

For example, as shown in FIGS. 14 and 17, there is possible an embodiment in which the rear connection member 131 protruding upward from the rear support member 130 is provided and connects the vertical support member 400 and the rear connection member 131 to each other.

The vertical support member 400 coupled to one side of the breast cover member 200A or 200B provided with a cutout portion 300 may be provided in a structure in which the binding level thereof can be adjusted. When the breast that is not subjected to radiation treatment is accommodated in the breast cover part, the vertical support member 400 may be adjusted to a loose binding level so that the accommodated breast can smoothly move down outward into the cutout portion 300 through free falling under gravity.

In contrast, in the case where the breast that is subjected to radiation treatment is accommodated in the breast cover part, it is preferable that the vertical support member 400 is tightly bound at a necessary level in order to fix the position and posture of the breast to be treated.

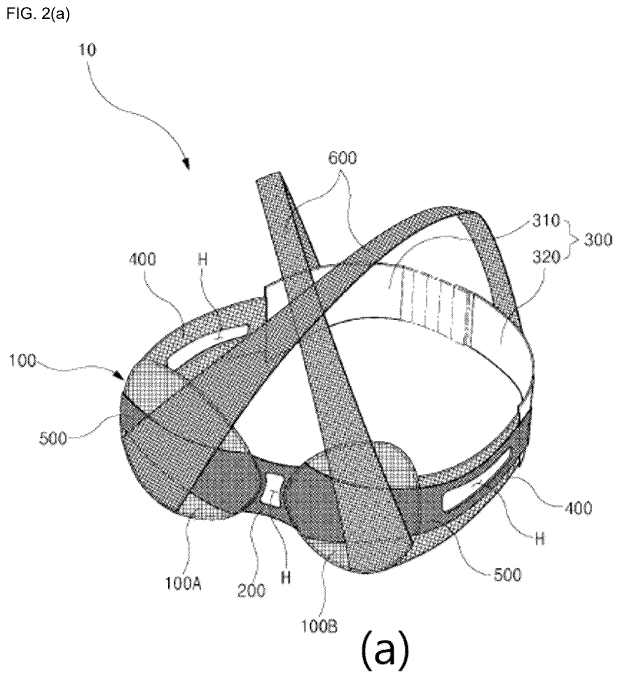

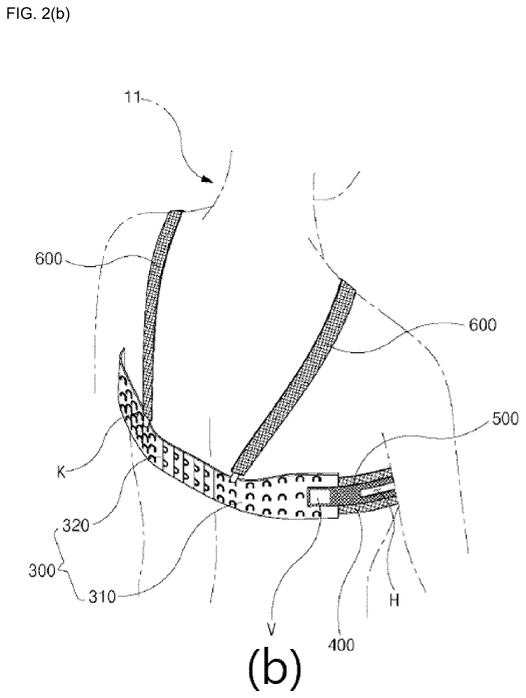

The binding structure according to the present invention may be provided in various forms. In an embodiment, the structure in which the binding level can be adjusted may be provided as a Velcro binding structure or a hook binding structure. FIG. 2(b) shows one example of a stepwise hook binding structure.

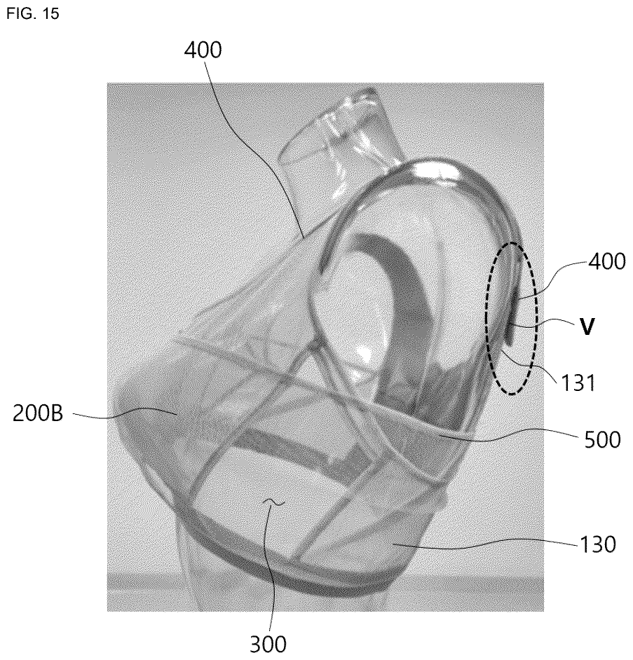

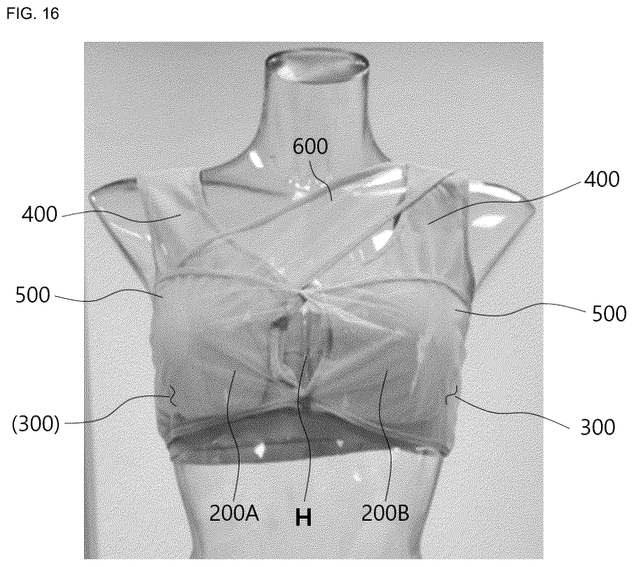

FIG. 17 shows an embodiment in which an end of the vertical support member 400 according to the present invention is bound to a Velcro fastening portion V that is provided on a rear connection member 131 protruding from a rear support member 130. FIG. 17(b) is an enlarged view of the dotted circular portion of FIG. 17(a). The dotted circular part of FIG. 17a corresponds to the dotted circular part of FIG. 15.

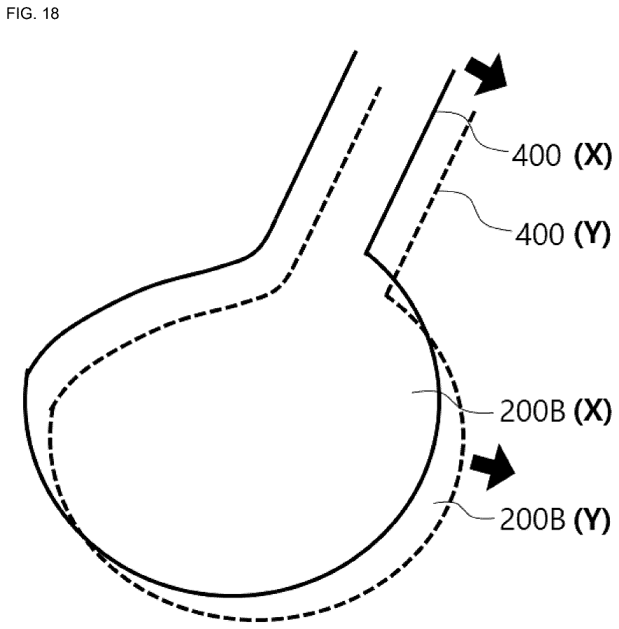

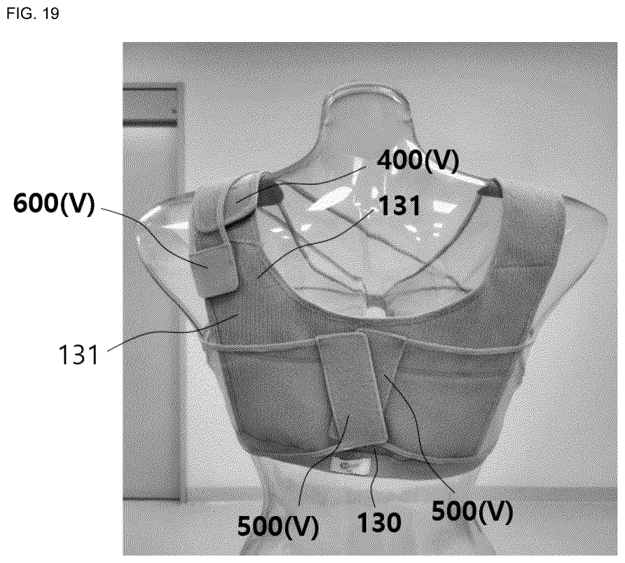

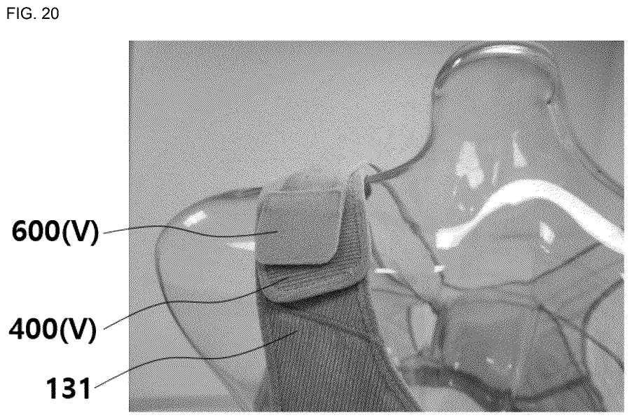

FIG. 18 shows a state in which one breast cover part 200B becomes loose as it changes from state (X) of FIG. 17 to state (Y) thereof.

As shown in FIG. 17, Velcro fastening portions V may be provided at the end of the vertical support member 400 and the end of the rear connection member 131, respectively, in order to face each other.

The state (X) of FIG. 17 is a state in which the end of the vertical support member 400 is lowered to the lower side of the Velcro fastening portion V of the rear connection member 131 and is bound to the lower side in a Velcro fastening manner. The state (Y) of FIG. 17 is a state in which the end of the vertical support member 400 is raised to the upper side of the Velcro fastening portion V of the rear connection member 131 and is bound to the upper side in a Velcro fastening manner.

When the vertical support member 400 is tightly bound to the rear connection member 131 as shown in the state (X) of FIG. 17, the breast cover part 200B coupled to the vertical support member 400 moves upward as shown in the state (X) of FIG. 18, so that the breast can be supported to prevent the breast from falling downward.

In contrast, when the vertical support member 400 is loosely bound to the rear connection member 131 as shown in the state (Y) of FIG. 17, the breast cover part 200B coupled to the vertical support member 400 moves downward as shown in the state (Y) of FIG. 18, so that the breast can easily fall freely under gravity.

For example, depending on the patient's body size or breast size, the extent to which the breast that is not subjected to radiation treatment moves into the cutout portion 300 may vary. Accordingly, a practitioner may set the maximum extent, to which the breast not subjected to radiation treatment moves into the cutout portion 300, by adjusting a Velcro binding level through the Velcro portion provided at the end of the vertical support member 400. The adjustment of the Velcro binding level may be performed using an oil-based pen so that it remains the same for every setup.

The diagonal support member 600 according to the present invention will be described below.

The diagonal support member 600 according to the present invention may be provided on at least one of a pair of breast cover parts 200A and 200B with one end thereof coupled to a side portion 120 of one breast cover part and the other end thereof coupled to any one of the vertical support member 400 connected to the other breast cover part, the rear support member 130, and the rear connection member 131 protruding upward from the rear support member 130.

In the case of the first main embodiment described above, there may be further provided the diagonal support member 600 one end of which is coupled to the side portion 120 of the body part 100 on one breast cover part side and the other end of which is coupled to the vertical support member connected to the other breast cover part.

In the case of the second main embodiment described above, there may be further provided the diagonal support member 600 one end of which is coupled to the side portion 120 of the body part 100 on one breast cover part side and the other end of which is coupled to the rear support member 130 of the body part 100 on the other breast cover part side.

A through hole H according to the present invention will be described below.

The through hole H according to the present invention is provided in the central portion of the body part 100 so that the two breasts accommodated in the bra can be separated and moved and fixed more precisely. A part or all of the one end of the lateral support member 500 may be coupled to the edge of the through hole H.

For example, despite the presence of the lateral support member 500 and the diagonal support member 600, there may be cases where the breast is fixed to the bra, so that the movement and support of the breast are not performed smoothly. In this case, the through hole H according to the present invention facilitates the free movement, support, and fixation of each breast, and enhances the degree of freedom of the lateral support member 500 and the diagonal support member 600, thereby reinforcing a bending function.

As shown in FIG. 4, when a lateral force is applied to the body part 100, the bending function is exerted due to the through hole H, so that the body part 100 can be extended further in the lateral direction.

In addition, for example, it may be possible to move and support the breast subjected to radiation treatment in one breast cover part 200A and the breast not subjected to radiation treatment in the other breast cover part 200B through the through hole H. It may be possible to support and fix the breast subjected to radiation treatment, thereby increasing the reproducibility and accuracy of the treatment, and it may also be possible to separate the breast not subjected to radiation treatment from the radiation treatment area and fix the breast, thereby protecting the breast from scattered rays.

The optimal geometrical posture for radiation treatment may be generated, and this setup may be reproducibly maintained for every treatment. Generally, radiation treatment is performed 5 to 28 times depending on the treatment technique. Meanwhile, there arises a problem in that the flexible breast, which is the target of treatment, is set up differently because it keeps moving for each treatment. Accordingly, the reproducibility of the setup may be secured via the bra according to the present invention. In the bra according to the present invention, the through hole H may strengthen the role of bending, which is a main function. Through this, radiation may be accurately delivered to the tumor breast that is subjected to radiation treatment, and the transmission of scattered radiation to the breast that is not subjected to radiation treatment may be prevented as much as possible by separating and fixing the breast not subjected to radiation treatment.

An air-equivalent material will be described below.

An air-equivalent material refers to a material having the same effective atomic number as air and exhibiting the same ionization to radiation. Examples of the air-equivalent material include graphite, paper, and specific plastics.

In the first main embodiment of the present invention, at least one of the body part 100, the pair of breast cover parts 200, and the lateral support member 500 may be made of an air-equivalent material woven in a mesh shape.

In the second main embodiment of the present invention, the lateral support member 400 may be made of an air-equivalent material woven in a mesh shape.

In the third main embodiment of the present invention, the diagonal support member 600 may be made of an air-equivalent material woven in a mesh shape.

In one embodiment, the breast cover parts 200 may be made of carbon fiber woven in a mesh shape. When the breast cover parts 200 are made of carbon fiber, carbon fiber exhibits the same ionization as air with respect to radiation, so that it does not affect the radiation dose radiated to the lesion area of the breast. Furthermore, when the breast cover parts 200 are woven in a mesh shape, there is an advantage in that the breast cover parts 200 are easily deformed when an external force is applied.

Meanwhile, as a specific embodiment according to the present invention, the following invention provided with at least one cutout portion, at least one lateral support member, at least one lateral support member, and at least one diagonal support member may be implemented.

The present invention provides a breast fixation bra for radiation treatment provided with a pair of breast cover parts having at least one cutout portion, the breast fixation bra including: a body part 100 provided to cover the breasts in the lateral direction; a pair of breast cover parts 200A and 200B provided to protrude on both sides of the central portion 110 of the body part 100 in order to correspond to the shapes of the breasts; at least one cutout portion 300 provided in a shape in which at least a portion is cut out from a corresponding one of the breast cover parts 200A and 200B; at least one vertical support member 400 provided on at least one of the pair of breast cover parts 200A and 200B with one end thereof coupled to one side of the breast cover part 200A or 200B and the other end thereof coupled to any one of the rear support member 130 of the body part 100 and a rear connection member protruding upward from the rear support member 130 via the shoulder; at least one lateral support member 500 provided across at least one of the breast cover parts 200A and 200B with one end thereof coupled to the central portion 110 of the body part 100 and the other end thereof coupled to a side portion 120 or the rear support member 130 of the body part 100, and configured to support the breast accommodated in the cutout portion 300; and at least one diagonal support member 600 provided across at least one of the pair of breast cover parts 200A and 200B with one end thereof coupled to a side portion 120 of the breast cover part 200A or 200B and the other end thereof coupled to any one of the vertical support member 400 coupled to the other breast cover part 200A or 200B, the rear support member 130, and the rear connection member 131 protruding upward from the rear support member 130.

The embodiments described herein and the accompanying drawings are merely illustrative of some of the technical spirit included in the present invention. Therefore, the embodiments disclosed herein are not intended to limit the technical spirit of the present invention, but are intended to describe the technical spirit of the present invention. Accordingly, it will be apparent that the scope of the technical spirit of the present invention is not limited by these embodiments. All modifications and specific embodiments that may be easily inferred within the scope of the technical spirit included in the specification and accompanying drawings of the present invention by those skilled in the art should be interpreted as being included in the scope of the present invention.

What is claimed is:

1. A breast fixation bra for radiation treatment provided with a pair of breast cover parts having at least one cutout portion, the breast fixation bra comprising:
a body part provided to cover breasts in a lateral direction;
a pair of breast cover parts provided to protrude on both sides of a central portion of the body part in order to correspond to shapes of the breasts;
at least one cutout portion provided in a shape in which at least a portion is cut out from a corresponding one of the breast cover parts; and
at least one lateral support member provided across at least one of the breast cover parts with one end thereof coupled to the central portion of the body part and a remaining end thereof coupled to a side portion or the rear support member of the body part, and configured to support a breast accommodated in the cutout portion.

2. The breast fixation bra of claim 1, further comprising:
at least one vertical support member provided on at least one of the pair of breast cover parts with one end thereof coupled to one side of the breast cover part and a remaining end thereof coupled to any one of the rear support member of the body part and a rear connection member protruding upward from the rear support member via a shoulder.

3. The breast fixation bra of claim 1, further comprising:
at least one diagonal support member provided across at least one of the pair of breast cover parts with one end thereof coupled to a side portion of the breast cover part and a remaining end thereof coupled to any one of the vertical support member coupled to the remaining breast cover part, the rear support member, and a rear connection member protruding upward from the rear support member.

4. The breast fixation bra of claim 1, wherein the lateral support member comprises lateral support members that are symmetrically arranged across the breast cover parts, respectively.

5. The breast fixation bra of claim 1, wherein the lateral support member comprises a plurality of lateral support members that are provided across a preset one of the breast cover parts in an overlapping manner.

6. The breast fixation bra of claim 5, wherein, when two lateral support members are provided across one of the breast cover parts in an overlapping manner,
one end of the lateral support member provided on a lower side is coupled to the side portion or rear support member of the body part, and
one end of the lateral support member provided on an upper side is coupled to the rear support member of the body part.

7. The breast fixation bra of claim 1, wherein the cutout portion is cut out along an overall breast parenchyma line.

8. The breast fixation bra of claim 1, wherein the cutout portion is cut out along an overall breast parenchyma line, but at least a portion thereof is cut out through a portion outside the breast parenchyma line.

9. The breast fixation bra of claim 1, wherein the cutout portion has an area outside a center line of each of the breast cover parts that is larger than an area inside the center line.

10. The breast fixation bra of claim 1, wherein the cutout portion does not include a portion corresponding to a nipple.

11. The breast fixation bra of claim 2, wherein:
the vertical support member is provided in a structure in which a binding level can be adjusted, and
when a breast accommodated in the breast cover part is moved, the vertical support member is adjusted to a loose binding level so that the accommodated breast can smoothly move down outward into the cutout portion through free fall under gravity.

12. The breast fixation bra of claim 11, wherein the structure in which the binding level can be adjusted is any one of a Velcro binding structure and a hook binding structure.

13. The breast fixation bra of claim 1, wherein a through hole is provided in the central portion of the body part, and allows both the breasts accommodated in the bra to be separated, and moved and then fixed.

14. The breast fixation bra of claim 13, wherein a part or all of the one end of the lateral support member is coupled to an edge of the through hole.

15. The breast fixation bra of claim 1, wherein at least one of the body part, the pair of breast cover parts, and the lateral support member is made of an air-equivalent material woven in a mesh shape.

16. The breast fixation bra of claim 2, further comprising:
at least one diagonal support member provided across at least one of the pair of breast cover parts with one end thereof coupled to a side portion of the breast cover part and a remaining end thereof coupled to any one of the vertical support member coupled to the remaining breast cover part, the rear support member, and a rear connection member protruding upward from the rear support member.

17. The breast fixation bra of claim 2, wherein the vertical support member is made of an air-equivalent material woven in a mesh shape.

18. The breast fixation bra of claim 3, wherein the diagonal support member is made of an air-equivalent material woven in a mesh shape.

19. A breast fixation bra for radiation treatment provided with a pair of breast cover parts having at least one cutout portion, the breast fixation bra comprising:
a body part provided to cover breasts in a lateral direction;
a pair of breast cover parts provided to protrude on both sides of a central portion of the body part in order to correspond to shapes of the breasts;
at least one cutout portion provided in a shape in which at least a portion is cut out from a corresponding one of the breast cover parts;
at least one vertical support member provided on at least one of the pair of breast cover parts with one end thereof coupled to one side of the breast cover part and a remaining end thereof coupled to any one of a rear support member of the body part and a rear connection member protruding upward from the rear support member via a shoulder;
at least one lateral support member provided across at least one of the breast cover parts with one end thereof coupled to the central portion of the body part and a remaining end thereof coupled to a side portion or the rear support member of the body part, and configured to support a breast accommodated in the cutout portion; and
at least one diagonal support member provided across at least one of the pair of breast cover parts with one end thereof coupled to a side portion of the breast cover part and a remaining end thereof coupled to any one of the vertical support member coupled to the remaining breast cover part, the rear support member, and the rear connection member protruding upward from the rear support member.
